(19) United States
(12) Patent Application Publication
(10) Pub. No.: US 2003/0047009 A1
(43) Pub. Date: Mar. 13, 2003

(54) Digital callipers

(76) Inventor: Walter L. Webb, Hendersonville, NC (US)

Correspondence Address: John B Hardaway III, Nexsen Pruet Jacobs & Pollard, PO Box 10107, Greenville, SC, 29603, US

(21) Appl. No.: 10/220,081

(22) PCT Filed: Feb. 26, 2001
(86) PCT No.: PCT/US01/05905

(51) Int. Cl. G01B 3/00; G01L 1/00; A61B 5/107

(52) U.S. Cl. 73/862.541; 33/501.02; 33/512

(57) ABSTRACT

Described is a calliper for measuring a size of an object and a force applied to the object to measure the size wherein the calliper comprises a scale support (2) comprising a fixed measuring finger (14). A movable mount (1) is slidably attached to the scale support wherein the movable mount comprises a second measuring finger (16) wherein the size is a distance between the fixed measuring finger and the second measuring finger when the fixed measuring finger and the second measuring finger contact the object. A detector (25), attached to the movable mount is capable of determining the distance between the second measuring finger and the fixed measuring finger. A force arm (10) is slidably attached to the movable mount and a sensor (11) attached to both the force arm and the movable mount such that the sensor detects a force applied to the force arm. A processor (55) is provided which is capable of receiving the distance and converting the distance to a displayable size element and capable of receiving the force and converting the force to a displayable force element. The displayable size element and displayable force element are displayed.

### FIELD OF THE INVENTION

This invention relates to apparatus and method for accurate measurement of size and the force used to determine the size. More specifically, this invention relates to calipers which are capable of measuring both size and force applied to an object.

### BACKGROUND OF THE INVENTION

Calipers have long been known and used for determining the thickness of an object. Typically a caliper comprises two fingers which are brought into contact with the outer extent of an object. The distance between the fingers is determined as the thickness, or size, of the object. In a similar manner fingers can be used which measure a void in a similar fashion by inserting the fingers in the void and widening them until they each contact the walls of the void.

One deficiency with the use of calipers is the variation in pressure which can be applied to the fingers and the differences in measurement which can occur. This is particularly true when a soft material is being measured such as some plastics, some soft metals, wood, styrofoam and the like. One particular application is the measurement of the thickness of a wire as a quality control parameter during manufacture. If the measurement is taken while the wire is somewhat malleable the measurement of thickness could be altered by one measurer applying a high level of pressure on the fingers thereby partially indenting the metal while another measurer applys a low level of pressure to the finger such that the metal is not indented. It is also often a desire in the art to provide a thickness/pressure profile to determine degree of curing and the like.

The caliper described in U.S. Pat. No. 4,606,128 utilizes a linear potentiometer to determine the pressure applied to the article being measured. The device is set at a predetermined pressure and is blocked from exceeding the pressure. There is no ability to achieve a profile of pressure and size since a fixed pressure is utilized.

U.S. Pat. No. 4,188,727 describes a caliper with an analog pressure measurement device based on a spring mechanism. As pressure is applied to the measuring jaw a pointer deflects to indicate the pressure. While operative, the spring loading is susceptible to corrosion and variations in spring strength due to temperature fluctuations. This is undesirable for accurate measurement since the variations in individual calipers over time can be large. Furthermore, the difference between different calipers can be extreme since storage conditions, and care, can dictate the quality of the springs.

U.S. Pat. No. 4,389,783 describes a caliper which establishes a constant pressure independently of the intention of the measurer. While certain advantages are offered there is no ability for the measurer to utilize the relationship between pressure and size since this information is not available from the calipers described.

The present invention provides the long sought device which can determine the size of an item at a given pressure easily and reliably.

### SUMMARY OF THE INVENTION

It is an object of the present invention to provide a caliper which can determine the size of an object and the pressure applied to determine the size.

It is another object of the present invention to provide a caliper which can be used to measure a size at a predetermined pressure applied and to report both the pressure applied and the size of the item.

A particular feature of the present invention is the avoidance of the use of springs which are susceptible to changes in response due to corrosion and temperature.

These and other advantages, as will be realized, are provided in a caliper for measuring a size of an object and a force applied to the object to measure the size wherein the caliper comprises a scale support comprising a fixed measuring finger. A movable mounting is slidably attached to the scale support wherein the movable mounting comprises a second measuring finger wherein the size is a distance between the fixed measuring finger and the second measuring finger when the fixed measuring finger and the second measuring finger contact the object. A detector, attached to the movable mounting is capable of determining the distance between the second measuring finger and the fixed measuring finger. A force arm is slidably attached to the movable mounting and a force gauge is attached to both the force arm and the movable mounting such that the force gauge detects a force applied to the force arm. A processor is provided which is capable of receiving the distance and converting the distance to a displayable size element and capable of receiving the force and converting the force to a displayable force element. The displayable size element and displayable force element are displayed.

Another embodiment is provided in a caliper for measuring a size of an object and measuring the force applied to the object to measure the size. The caliper comprises a scale support comprising a fixed measuring finger and a scale. A movable mounting is slidably attached to the scale support. The movable mounting comprises a second measuring finger wherein the size is a distance between the fixed measuring finger and the second measuring finger. A detector is attached to the movable mounting and is capable of reading the scale and determining a position of the movable mounting relative to the scale support. A force arm is slidably attached to the movable mounting. A force gauge is attached to both the force arm and the movable mounting. The force gauge detects an applied force on the force arm. A processor converts the position to a distance between the fixed measuring finger and the second measuring finger to a displayable distance and converts the applied force to a displayable force element. The displayable distance and the displayable force element preferably are displayed.

Another embodiment is provided in a caliper for measuring a size of an object and the force applied to the object to measure the size. The caliper comprises a scale support comprising a fixed measuring finger. A movable mounting is slidably attached to the scale support. The movable mounting comprises a second measuring finger wherein the size is a distance between the fixed measuring finger and the second measuring finger when the fixed measuring finger and the second measuring finger contact the object. A detector, capable of determining a position of the movable mounting relative to the scale support. is also attached to the movable mounting. A force arm is slidably attached to the movable mounting and a strain gauge attached to both the force arm and the movable mounting wherein the force gauge deflects when a force is applied to the force arm.

A particularly preferred embodiment is provided in a method for determining the size of an object with a caliper. The method comprising the steps of:

a) contacting opposing sides of the object with a first measuring finger and a second measuring finger of a caliper; and

b) monitoring a force applied to the object by the first measuring finger and the second measuring finger. The caliper comprises a scale support with the first measuring finger attached thereto. A movable mounting is slidably attached to the scale support. The movable mounting comprises a second measuring finger. A detector capable of determining a position of the movable mounting relative to the scale support is attached to the movable mounting. A force arm is slidably attached to the movable mounting. A strain gauge is attached to both the force arm and the movable mounting wherein the force gauge deflects proportional to the force.

Another embodiment is provided in a caliper for measuring a size of an object and the force applied to the object. The caliper comprises a scale support comprising a fixed measuring finger and a scale. A movable mounting is slidably attached to the scale support wherein the movable mounting comprises a second measuring finger wherein the size is a distance between the fixed measuring finger and the second measuring finger when the fixed measuring finger and the second measuring finger are in contact with the object. a detector capable of reading the scale and determining a position of the movable mounting relative to said scale support is attached to the movable mounting. A force arm is slidably attached to the moving mount and a strain gauge is attached to both the force arm and the movable mounting wherein the force gauge detects a force applied to the force arm when the fixed measuring finger and the second measuring finger contact the object. A processor is provided which is capable of converting the position to a distance between the fixed measuring finger and the second measuring finger to a displayable distance and converting the force to a displayable force element and providing the information to a display.

Yet another embodiment is provided in a caliper for measuring the size of an object and the force applied to the object. The caliper comprises a scale support comprising a fixed measuring finger and a scale. A movable mounting is slidably attached to the scale support wherein the movable mounting comprises a second measuring finger wherein the size is a distance between the fixed measuring finger and the second measuring finger when the fixed measuring finger and the second measuring finger contact the object. A detector is provided which is capable of reading the scale by capacitive displacement and determining a position of the movable mounting relative to the scale support. A force arm is slidably attached to the movable mounting. A strain gauge is attached to both the force arm and the movable mounting wherein the force gauge detects a force applied to the force arm when the fixed measuring finger and the second measuring finger contact the object. A processor converts the position to a distance between the fixed measuring finger and the second measuring finger to a displayable distance and converts the force to a displayable force element and they are displayed.

### BRIEF DESCRIPTION OF THE DRAWINGS

FIG. 1 is a top view of the calipers of the present invention.

FIG. 2 is a perspective view of the calipers of the present invention.

FIG. 3 is a side view of the calipers of the present invention.

FIG. 4 is a perspective exploded view of the front side of the calipers of the present invention.

FIG. 5 is a perspective exploded view of the back side of the calipers of the present invention.

### DETAILED DESCRIPTION OF THE INVENTION

The invention will be described in reference to the preferred embodiments which are set forth in the drawings and descriptions. Throughout the drawings, and descriptions thereof, similar elements are numbered accordingly.

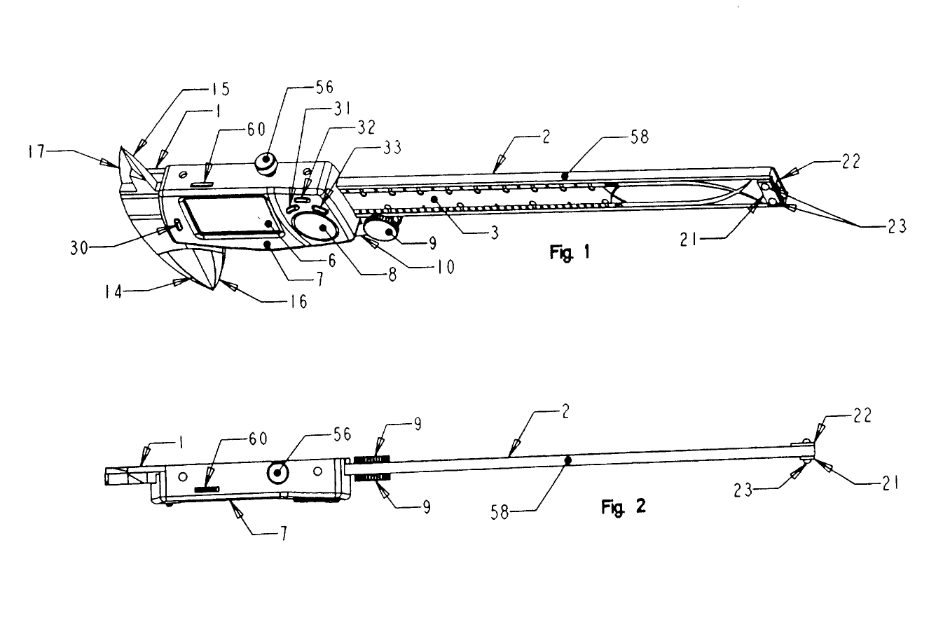

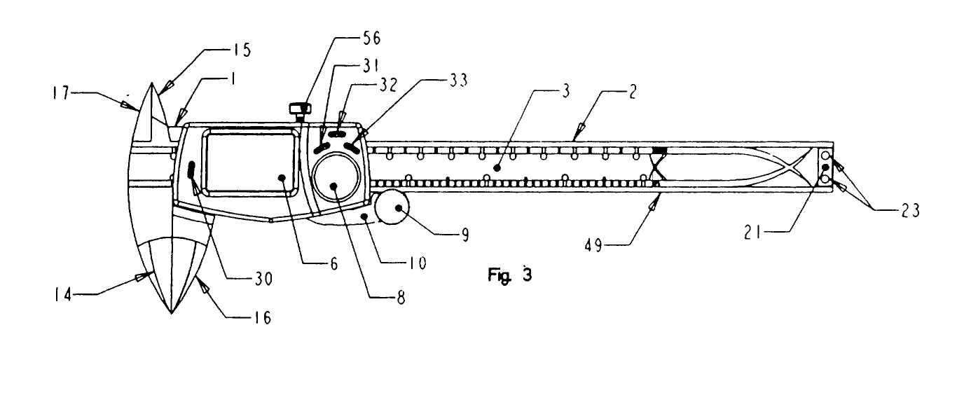

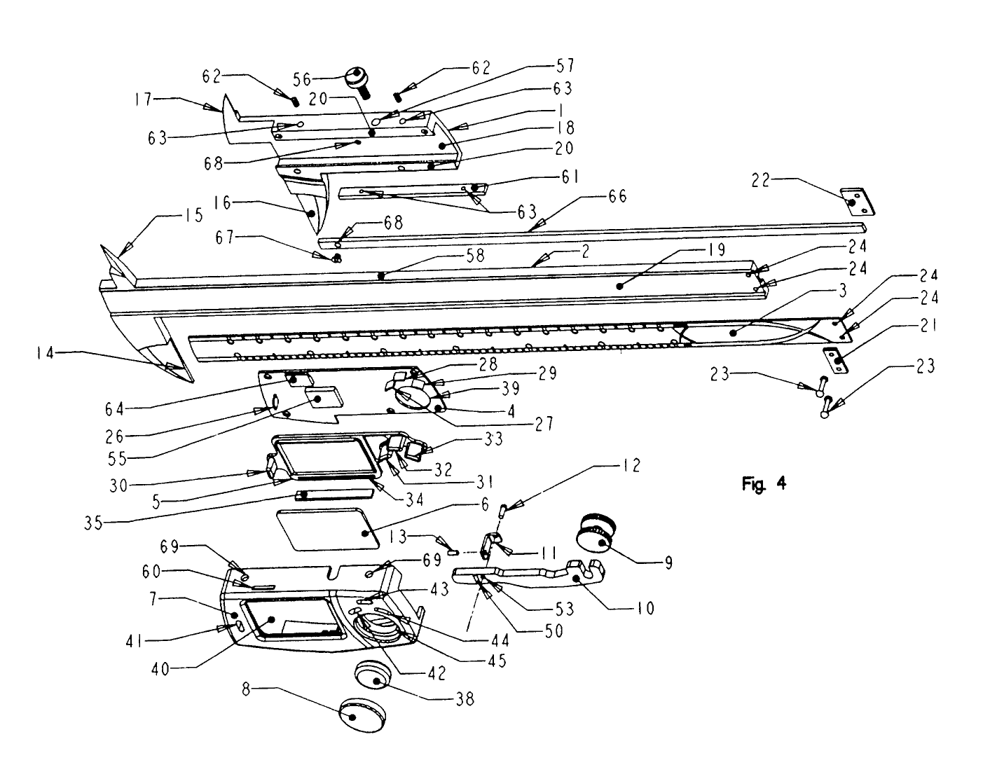

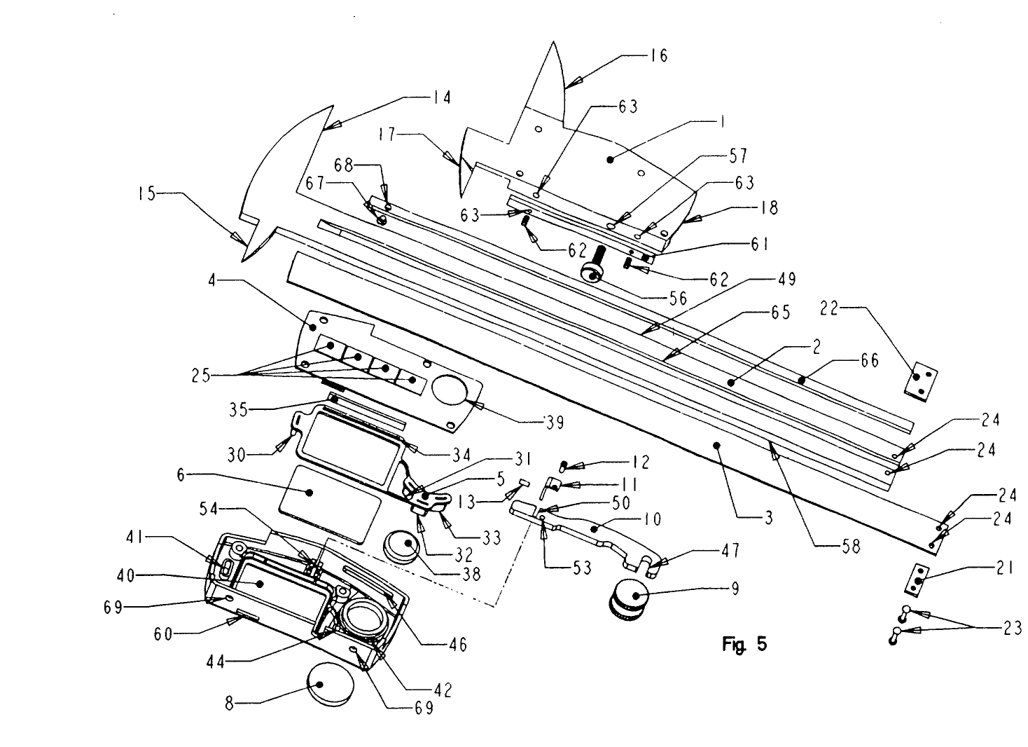

FIG. 1 is a top side view of the calipers of the present invention. FIG. 2 is a perspective view of the calipers of FIG. 1. FIG. 3 is a side view of the calipers of FIG. 1. FIG. 4 is an expanded view illustrating the various internal components and FIG. 5 is a back side view of the components of FIG. 4.

The calipers comprise a scale unit support, 2, which serves as a basic structural element of the calipers. The scale unit support, 2, comprises a fixed external measuring finger, 14, attached on one end thereof and an optional fixed internal measuring finger, 15, attached to the same end of the scale unit support but opposite to the fixed external measuring finger. As will be realized the fixed external measuring finger, 14, and fixed internal measuring finger, 15, both are fixed relative to the scale unit support, 2. Slidably attached to the scale unit support, 2, is a moving mount, 1. The moving mount, 1, comprises a moving external measuring finger, 16, and a moving internal measuring finger, 17, both of which move in concert with the moving mount, 1. As would be apparent to one of ordinary skill in the art the external measuring fingers work in concert to engage the external surface of the item to be measured and the distance there between is the size or thickness of the item. In a similar fashion, the internal measuring fingers work in concert to engage the interior surface of a void to be measured and when both internal measuring fingers are in contact with the inner walls of a void the distance there between corresponds to the separation across the void. In the discussions that follow the distance between fingers refers to the distance between the surface of the fingers which contact the object being measured.

The moving mount, 1, comprises a channel, 18, wherein the scale unit support, 2, traverses. The channel is preferably sized to allow slightly restricted lateral movement of the channel along the length of the scale unit support with minimal movement perpendicular to the scale unit support. The scale unit support, 2, comprises a scale slot, 19, which securely receives a scale, 3, the significance and details of which will be described in further detail below.

A printed circuit board, or PCB, 4, mounted to the exterior surface, 20, of the moving mount, 1, encases the scale unit support, 2, and attached scale, 3. The back side of the printed circuit board, 4, (see FIG. 5) comprises at least one detector, 25, capable of detecting the position of the moving mount relative to the scale unit support by correlation to the scale, 3. Redundant detectors can be utilized as would be realized in the art. The detector is preferably a coupling electrode which couples with the scale, preferably by capacitive displacement, to precisely determine the position of the moving assembly relative to the fixed assembly. The use of coupling electrodes and scales in this manner are well known in the art and further elaboration herein is not necessary. An exemplary capacitive displacement measuring system and electronic circuitry to operate such a device is described in U.S. Pat. No. 4,586,260 which is included herein by reference thereto.

The printed circuit board, 4, comprises contacts, 26-29, for controlling the electronics of the caliper. At least one contact, 26, preferably allows the caliper to be turned on and off but this may be eliminated if an alternate power source such as a solar cell is utilized. The control contacts, 27-29, are utilized to alter the mode of operation and/or display. The mode may be changed to different units, such as inches versus mm, pounds versus grams and the like. The mode may also be changed to provide for various alerts such as flashing display or an audible alarm when the proper pressure is reached. Features may also be temporarily disabled if, for example, the calipers are being used in a condition were pressure is not critical and therefore the display can be limited to increase battery life. Other modes which may be activated by the contacts include averaging algorithms which collect data over a predetermined time period and average the results; smoothing or dampening algorithms to decrease data spikes due to rapid movement and the like; display alterations such as distance to pressure profiles, pressure or distance histories, etc. Integral to, or attached to, the printed circuit board, 4, is a silicon button assembly, 5, which further comprises optional button caps, 30-33, each of which corresponds to one contact, 26-29. Pressing the button cap closes the contact thereby communicating with the electronic circuitry as previously described. The silicon button assembly, 5, comprises a passage slot, 34, thereby allowing a carbon contact strip, 35, to be in operative contact with both the printed circuit board, 4, and with a display, 6. A battery, 38, is preferably received in a battery hold, 39, in the printed circuit board, 4.

The printed circuit board, 4, silicon button assembly, 5, display, 6, battery, 38, and associated attachments are encased by a scale housing, 7. The scale housing comprises an LCD slot, 40, for receiving the display and cap slots, 41-44, for receiving the button caps, 30-33. A battery passage, 45, and associated battery cap, 8, allow easy access to the battery for changing. In an alternate embodiment a solar cell may be used as a power source. This is well developed technology and further elaboration is not necessary.

At the terminal end of the scale unit support is a stop comprising a stop front plate, 21, and stop rear plate, 22. The stop front plate and stop rear plate are secured to the terminal end of the scale unit support by elongated members, 23, such as rivets, threaded assemblies or the like. The elongated members are inserted through securing voids, 24, in the scale unit support, 2, and scale, 3.

The bottom side of the scale housing, 7, comprises a force arm slot, 46, for receiving a force arm, 10. The force arm, 10, freely floats parallel to the long axis of the apparatus within the force arm slot. The outer end of the force arm comprises a wheel slot, 47, for receiving a guide wheel, 9. The guide wheel frictionally engages with the lower surface, 49, of the scale unit support. The guide wheel is rotated to persuade the moving mount, 1, and associated parts, to traverse back and forth along the length of the scale unit support as known in the art. The inner end of the force arm, 10, comprises a sensor slot, 50, which receives a sensor, 11. The sensor is secured to the force arm by an sensor mounting element, 12, such as a screw or rivet, and associated receiving void, 53. The sensor is fixedly attached to the scale housing, 7, by receipt in a sensor bracket, 54. A sensor holding pin, 13, secures the sensor within the sensor bracket. As force is applied on the force arm the amount of force is detected by the sensor and a signal related to the amount of force is transmitted to the printed circuit board, 4, by any manner known in the art. As the measuring fingers are moved into contact with the object being measured there is minimal deflection on the sensor thus no force is displayed. After the measuring fingers contact the object any additional force on the force arm will cause deflection of the sensor. The force on the sensor translates to the force applied to the object.

The force required to deflect the sensor in an amount sufficient to generate a signal is preferably higher than the force required to move the moving mount and associated elements along the scale unit support. Furthermore, the recovering force of the sensor is preferably higher than that required to move the moving mount and associated elements along the scale unit support. The recovery force is that force that the sensor element imparts to return to rest condition. Therefore, if the calipers are placed on an object with a certain level of force the sensor will return the calipers to a position of neutral force unless impeded from so doing by the user physically holding the caliper in a position with the force being measured.

A processor, 55, preferably attached to the printed circuit board, 4, receives a signal from the detector, 25, and converts the signal to a displayable distance number representing the separation between the measuring fingers and communicates with the display, 6, to display the separation as a distance between measuring fingers. It would be apparent to one of ordinary skill in the art that the distance between fingers is the size of the object being measured. The process also receives a signal from the sensor, 11, indicating the force applied to the force arm when the object is in contact with the appropriate measuring arms. The processor converts the signal from the sensor to a displayable force number and communicates with the display to display the force. It would be apparent to one of ordinary skill in the art that correction for friction between the caliper elements may be required to accurately correlate the force applied at the sensor to the force applied at the object.

An optional lock button, 56, secures the moving mount in a specific position relative to the scale unit support. The lock button, 56, may be threaded and received in a threaded void, 57. A friction resistance bar, 61, is preferably attached to the movable mount, 1, between the moveable mount and the upper surface, 58, of the scale unit support, 2. The friction resist bar is preferably attached by set screws, 62, received in aligned voids, 63, which are accessible through set screw access voids, 69. The friction resistance bar, 61, is preferably constructed of a pliable metal such as copper. Other materials of construction are also contemplated such as metals, synthetic materials including plastics such as teflon®, or natural products. The lock button is rotated to adjust the tension between the friction resistance bar, 61, and the upper surface, 58, of the scale unit support. The ease with which the moving mount is moved is adjusted by friction. Other methods for engaging the lock button are within the scope of the invention including click lock similar to the mechanism in a retractable pen.

An optional interface port slot, 60, allows access to an optional data port, 64, which is preferably integral to the printed circuit board, 4. The data port, 64, allows the calipers to be connected to a computer for exchanging data collected, to program various components, or to provide a display. The interface may also be used for supplying power to the device.

The sensor is preferably a strain gauge which generates a signal proportional to the degree of bending of the strain gauge. Therefore, in the present application, the measuring fingers come into contact with the object being measured. Any additional force applied to the force arm will cause a deflection, or bending of the strain gauge. The more force applied to the force arm the higher the deflection and therefore the higher the measured force. The deflection is communicated to the processor as a magnitude and the magnitude of deflection is converted to a force for display. A particularly exemplary force gauge is available from the Micro-Measurements Division of Measurements Group, Inc. of Raleigh, N.C. In general, the strain gauge comprises a strain-sensitive foil grid which is held in place on the top surface of a flexible carrier, or backing. The bottom surface of the backing is adhesively bonded to a part, member, or structure, any load induced strains are transmitted through the backing to the grid. The strain gauge is not limiting with the exceptions of size and range which are chosen for the particular application. Other sensors, particularly linear sensors, can be utilized for the present invention. For example, a piezoelectric detector can be utilized by securing one side of the piezoelectric sensor to the force arm and the other side to the moving mount. As pressure is applied the piezoelectric sensor would generate a proportional signal as well known in the art of piezoelectric pressure sensors. An optical system can also be used wherein the force is determined as the degree of deflection of an optical fiber. The deflection is then measured by a light detector or other suitable means. The strain gauge is preferred due in part to cost, availability and ease with which they can be incorporated into a device which is easily and economically manufactured.

The detector, 25, and scale, 3, work in concert to accurately determine the position of the detector of the moving mount relative to the scale. Since the position of the scale is fixed relative to the fixed measuring fingers and the position of the detector is fixed relative to the measuring fingers of the moving mount the distance between the measuring fingers can be easily determined. The scale preferably comprises a plurality of lithographically etched thin flat electrically conductive elements, preferably copper, which are prepared in a conventional manner using photo resists and etchants. The detector preferably comprises a slider board with a slider pattern similarly etched on the side facing the scale such that the scale and slider board are in spaced opposition. The scale is preferably passive. The slider board preferably comprises active transmitting and sensing elements with provisions for electrical connection to other electrical components such as the processor and power source. The detector preferably determines the position relative to the scale by capacitive displacement.

The display is preferably a liquid crystal display due, in part, to the wide range of commercially available displays. The display preferably can display distance and force at the same time but a single display area can be used wherein distance and force are intermittently displayed. In a particularly preferred embodiment the display can display both distance and force and the units for each. It is further contemplated that other information can be displayed such as graphics and text. Particularly contemplated are graphical representations of force to distance profiles, force history as a function of other parameters such as time, etc.

In use the calipers can be used as standard calipers wherein the additional information regarding force applied can be observed. The calipers may also be used by observing the force and contacting an object with ever increasing force on the force arm until a predetermined level is reached at which point the distance, or size, is observed.

The invention has been described with particular emphasis on the preferred embodiments. The teachings herein would lead a skilled artisan to variations and alterations and design choices. The invention is not to be construed as limited by the preferred embodiment described herein but instead as set forth in the claims which follow.

What is claimed is:

1. A caliper for measuring a size of an object and a force applied to said object to measure said size wherein said caliper comprises:
a scale support comprising a fixed measuring finger;
a movable mounting slidably attached to said scale support wherein said movable mounting comprises:
a second measuring finger wherein said size is a distance between said fixed measuring finger and said second measuring finger when said fixed measuring finger and said second measuring finger contact said object;
a detector capable of determining said distance between said second measuring finger and said fixed measuring finger;
a force arm slidably attached to said movable mounting; and
a force gauge attached to said force arm and said movable mounting wherein said force gauge detects a force applied to said force arm;
a processor capable of receiving said distance and converting said distance to a displayable size element and receiving said force and converting said force to a displayable force element; and
a display capable of displaying said displayable size element and said displayable force element.

2. The caliper of claim 1 wherein said force gauge is a strain gauge.

3. The caliper of claim 1 wherein said scale support further comprises a second fixed measuring finger.

4. The caliper of claim 1 further comprising a scale attached to said scale support and said detector is capable of determining said distance by coupling with said scale.

5. The caliper of claim 4 wherein said detector detects said distance by capacitive displacement.

6. A caliper for measuring a size of an object and the force applied to said object to measure said size wherein said caliper comprises:
a scale support comprising a fixed measuring finger and a scale;
a movable mounting slidably attached to said scale support wherein said movable mounting comprises:
a second measuring finger wherein said size is a distance between said fixed measuring finger and said second measuring finger;
a detector capable of reading said scale and determining a position of said movable mounting relative to said scale support;
a force arm slidably attached to said movable mounting; and
a force gauge attached to said force arm and said movable mounting wherein said force gauge detects an applied force on said force arm;
a processor capable of converting said position to a distance between said fixed measuring finger and said second measuring finger to a displayable distance and converting said applied force to a displayable force element; and
a display capable of displaying said displayable distance and said displayable force element.

7. The caliper of claim 6 wherein said detector reads said scale by capacitive displacement.

8. The caliper of claim 6 wherein said force gauge is a strain gauge which deflects upon placement of said applied force on said force arm in an amount proportional to said applied force.

9. The caliper of claim 6 wherein said display is a liquid crystal display.

10. A caliper for measuring a size of an object and the force applied to the object to measure the size wherein said caliper comprises:
a scale support comprising a fixed measuring finger;
a movable mounting slidably attached to said scale support wherein said movable mounting comprises:
a second measuring finger wherein said size is a distance between said fixed measuring finger and said second measuring finger when said fixed measuring finger and said second measuring finger contact said object;
a detector capable of determining a position of said movable mounting relative to said scale support;
a force arm slidably attached to said movable mounting; and
a strain gauge attached to said force arm and attached to said movable mounting wherein said force gauge deflects when a force is applied to said force arm.

11. The caliper of claim 10 further comprising a processor capable of converting said position to a distance between said fixed measuring finger and said second measuring finger to a displayable distance and converting said applied force to a displayable force element.

12. The caliper of claim 11 further comprising a display capable of displaying said displayable distance and said displayable force element.

13. The caliper of claim 10 further comprising a depth gauge attached to said movable mounting.

14. A method for determining the size of an object with a caliper comprising the steps of:
a) contacting opposing sides of said object with a first measuring finger and a second measuring finger of a caliper;
b) monitoring a force applied to said object by said first measuring finger and said second measuring finger;
wherein said caliper comprises:
a scale support with said first measuring finger attached thereto; and
a movable mounting slidably attached to said scale support wherein said movable mounting comprises:
a second measuring finger;
a detector capable of determining a position of said movable mounting relative to said scale support;
a force arm slidably attached to said movable mounting; and
a strain gauge attached to said force arm and attached to said movable mounting wherein said force gauge deflects proportional to said force.

15. The method of claim 14 wherein said caliper further comprises a processor capable of converting said position to a distance between said fixed measuring finger and said second measuring finger to a displayable distance and converting said force to a displayable force element.

16. The method of claim 15 wherein said caliper further comprises a display capable of displaying said displayable distance or said displayable force element.

17. A caliper for measuring a size of an object and the force applied to the object to measure the size wherein said caliper comprises:
a scale support comprising a fixed measuring finger and a scale;
a movable mounting slidably attached to said scale support wherein said movable mounting comprises:
a second measuring finger wherein said size is a distance between said fixed measuring finger and said second measuring finger when said fixed measuring finger and said second measuring finger contact said object;
a detector capable of reading said scale and determining a position of said movable mounting relative to said scale support;
a force arm slidably attached to said moving mount; and
a strain gauge attached to said force arm and said movable mounting wherein said force gauge detects a force applied to said force arm when said fixed measuring finger and said second measuring finger contact said object;
a processor capable of converting said position to a distance between said fixed measuring finger and said second measuring finger to a displayable distance and converting said force to a displayable force element; and
a display capable of displaying said displayable distance and said displayable force element.

18. A caliper for measuring a size of an object and the force applied to the object to measure the size wherein said caliper comprises:
a scale support comprising a fixed measuring finger and a scale;
a movable mounting slidably attached to said scale support wherein said movable mounting comprises:
a second measuring finger wherein said size is a distance between said fixed measuring finger and said second measuring finger when said fixed measuring finger and said second measuring finger contact said object;
a detector capable of reading said scale by capacitive displacement and determining a position of said movable mounting relative to said scale support;
a force arm slidably attached to said movable mounting; and
a strain gauge attached to said force arm and said movable mounting wherein said force gauge detects a force applied to said force arm when said fixed measuring finger and said second measuring finger contact said object;
a processor capable of converting said position to a distance between said fixed measuring finger and said second measuring finger to a displayable distance and converting said force to a displayable force element; and
a display capable of displaying said displayable distance and said displayable force element.

19. A caliper for measuring a depth of a void and a force applied to measure said depth wherein said caliper comprises:
a scale support;
a movable mounting slidably attached to said scale support wherein said movable mounting comprises:
a depth gauge;
a detector capable of determining a position of said movable mounting relative to said scale support;
a force arm slidably attached to said movable mounting; and
a force gauge attached to said force arm and attached to said movable mounting wherein said force gauge deflects when a force is applied to said force arm.
